(19) United States
(12) Patent Application Publication
(10) Pub. No.: US 2003/0069477 A1
(43) Pub. Date: Apr. 10, 2003

(54) Surgical instrument

(75) Inventors: Ron Raisman, D.N. Be'er Yaacov (IL); Gabriel Savin, Rishon Le Zion (IL); Tzvika Vaknin, Rishon Le Zion (IL)

Correspondence Address: BROWDY AND NEIMARK, P.L.L.C., 624 NINTH STREET, NW, SUITE 300, WASHINGTON, DC, 20001-5303, US

(73) Assignee: Medic. NRG, D.N. Beer Yaacob (IL)

(21) Appl. No.: 09/970,035

(22) Filed: Oct. 4, 2001

(51) Int. Cl. A61B 1/32

(52) U.S. Cl. 600/220

(57) ABSTRACT

A surgical instrument of the type used for visualizing and carrying out different medical procedures within cavities in the body. The device comprises two rigid blades fitted for retracting tissue layers of the cavity thus foaming a path between the blades with a film of flexible and non elastic material ending between corresponding edges of the blades giving rise to side walls preventing collapse of tissue into the path between the blade.

### FIELD OF THE INVENTION

The present invention is generally concerned with a surgical instrument used for facilitating diagnostic and therapeutic procedures in body cavities. In particular, the invention is concerned with an improved speculum fitted with a circumferential retracting arrangement so as to prevent tissue collapse.

### BACKGROUND OF THE INVENTION AND PRIOR ART

A speculum is an apparatus used for visualizing internal cavities of an individual for carrying out various diagnostic and therapeutic procedures.

For example, a speculum is used for examining a woman's vagina. For that purpose, it is required that the speculum provide a clear path through which the internal organs may be visualized and through which some other equipment may be introduced for diagnostics and for carrying out surgical procedures.

A typical speculum comprises two blades being retractable from one another for widening the vagina (or other organ) for insertion of the blades therein, to thereby enable the inspection and insertion of instruments into the vagina through a path formed between the two blades.

However, since only two blades are used, it is often required to use other medical instruments such as retractors, for supporting the walls of the vagina and the labia from collapse into the path formed between the blades.

Several patents are concerned with the above problem and provide a speculum fitted with different arrangements for overcoming that problem, namely to prevent collapse of the walls into the path formed between the blade of the speculum. The following is a list of U.S. patents concerned with the above matter: U.S. Pat. Nos. 1,412,975, 1,614,065, 2,083,573, 2,374,863, 4,130,113; 5,509,893, 5,868,668, 6,024,696, 6,024,697, 6,036,638, 6,048,308. However, these patents are concerned with non-disposable devices which are complicated and expensive.

It is an object of the present invention to provide a speculum fitted with two rigid, retracting blades and side walls extending between the rigid blades for supporting walls of the organs into which the device is introduced and to prevent collapse of walls thereof into a path formed between the rigid blades.

### SUMMARY OF THE INVENTION

According to the present invention there is provided a surgical instrument of the type used for visualizing and carrying out different medical procedures within cavities in the body. The device comprises two rigid blades fitted for retracting tissue layers of the cavity thus forming a path between the blades with a film of flexible and non elastic material extending between corresponding edges of the blades giving rise to side walls preventing collapse of tissue into the path between the blade. The device is cheap and may thus be disposable.

In accordance with the present invention there is provided a surgical instrument for carrying out diagnostic and therapeutic procedures on interior organs of a body, the instrument comprising a rigid lower blade and a rigid upper blade, coupled so as to be spatially adjustable between a closed position and at least one open position, and a film of flexible material extending from corresponding side edges between the lower blade and the upper blade, which at an open position said film is tensioned thereby constituting two side walls of the instrument.

According to the invention, there is further provided a vaginal speculum comprising a rigid lower blade and a rigid upper blade, each comprising two side edges and being articulated in a manner so as to be spatially adjustable between a closed position and at least one open position; and two side walls made of a flexible material extending between corresponding side edges of the blades and sized such that at the open position said side walls are tensioned thus forming together with the blades a tubular portion, through which diagnostic and therapeutic procedures may be carried out and where at a closed position said walls are collapsed and received within a space between the lower blade and the upper blade.

The film of material may be a tubular sleeve enveloping both blades or a sheet enveloping one blade and secured at its ends at the other blade or two sheets each secured to both blades.

A sheet of material may be secured to the one or two blades by various means, e.g. by snap-type engagement, adhering, wedge-type locking arrangements, etc. It is advantageous that the sleeve and the securing means do not project beyond an outer surface of the blades and thus the blades may be formed with a receiving depression for accommodating the sleeve.

### BRIEF DESCRIPTION OF THE DRAWINGS

For better understanding the invention and to see how it may be cried out in practice, the invention will now be described with reference to the accompanying drawings, by way of example only, in which:

FIG. 1 is a side elevation of a speculum in accordance with the present invention, the speculum in its open position;

FIG. 2 is a perspective view of a speculum in accordance with another embodiment of the present invention, the speculum in its open position;

FIG. 3 is a longitudinal section taken along line III-III in FIG. 2, illustrating only the blades' portion of the speculum;

FIG. 4A is a perspective view of still another embodiment of a speculum in accordance with the present invention, one blade being partially sectioned;

FIG. 4B is a section through line IV-IV of the blade in the embodiment of FIG. 4A;

FIGS. 5A-5C arc sections corresponding with fat of FIG. 4B illustrating different embodiments for fixing a film of material to the blades of the speculum so as to constitute side walls thereof;

FIG. 6A is an isometric view of a speculum according to the invention in a closed position; and

FIG. 6B is a section view along line V-V in FIG. 5A.

### DETAILED DESCRIPTION OF SPECIFIC EMBODIMENTS

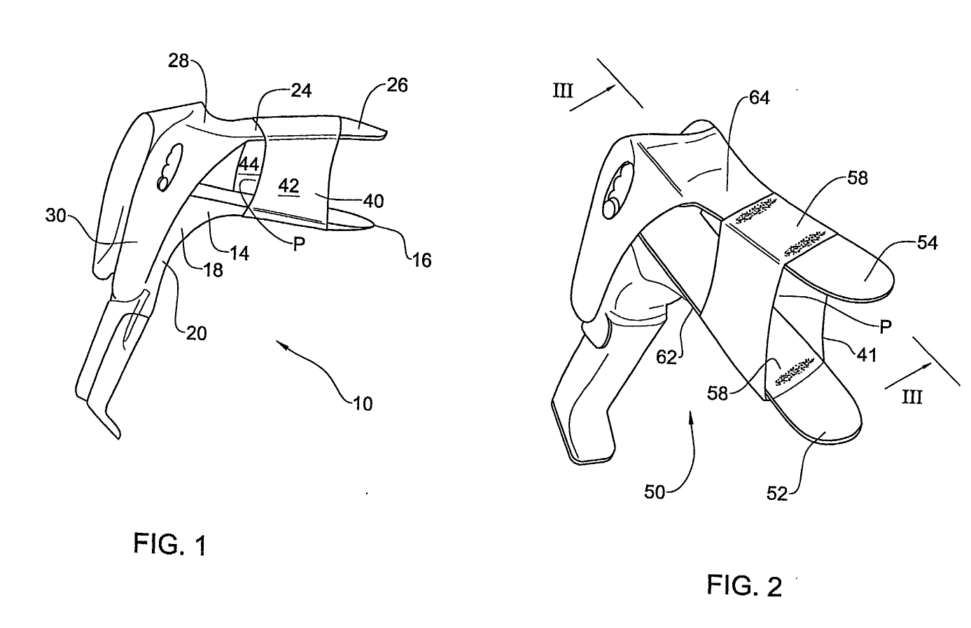

Attention is fist directed to FIG. 1 of the drawings illustrating a surgical instrument, namely a vaginal speculum generally designated 10.

Speculum 10 in the present embodiment is a disposable, two-piece device comprising a lower blade 14 extending between a distal end 16 and a proximal end 13 from which an integral handle 20 extends downwardly. An upper blade 24 has a distal end 26 and a proximal end 28 from which a manipulating handle 30 extends for achieving angular and elevational or spatial opening of the blades 14 and 24 as known per se. Blades 14 and 24 are made of rigid materials, possibly also transparent plastic material.

A film of material in the form of a sleeve 40 is fitted over the lower blade 14 and the upper blade 24 and is secured thereto. Film 40, in accordance with a particular embodiment of the invention is a transparent material which is non elastic though pliable.

The film 40 is sized such that when the speculum 10 is manipulated into its open position as illustrated in FIG. 1, e.g. by spatially displacing the upper blade 24 away from the lower blade 14 (typically manipulable by slidingly displacing handle 30 away from handle 20) the sleeve 40 becomes tensioned over edges of the lower blade 14 and upper blade 24, giving rise to constitution of two side walls 42 and 44 thus forming a tubular path P between the blades ad the sleeve. This arrangement ensures that when the device is in use and received within a cavity in the body, i.e. a woman's vagina, the blades 14 and 24 in conjunction with side walls 42 and 44 prevent the walls of the vagina and labia from collapse into the path P, whereby a gynecological examination or surgical procedure may be easier performed and further, protecting the vaginal region form pinching or otherwise injuring during the procedure.

The width of the film of material 40 may differ depending upon different speculum blades used and other particular constructions of the speculum. By some particular design the width of the film may span between about 3 to 10 cm.

In the embodiment of FIG. 1, side walls 42 and 44 are constituted by a single sheet of material 40 which is formed in the shape of a sleeve. However, as it will become apparent with reference to further embodiments illustrated hereinafter, the side walls may be constituted by a single sleeve of material enveloping only one blade and secured at both ends to the other blade, or by two film members, each secured at its respective ends to the lower and upper blades, respectively.

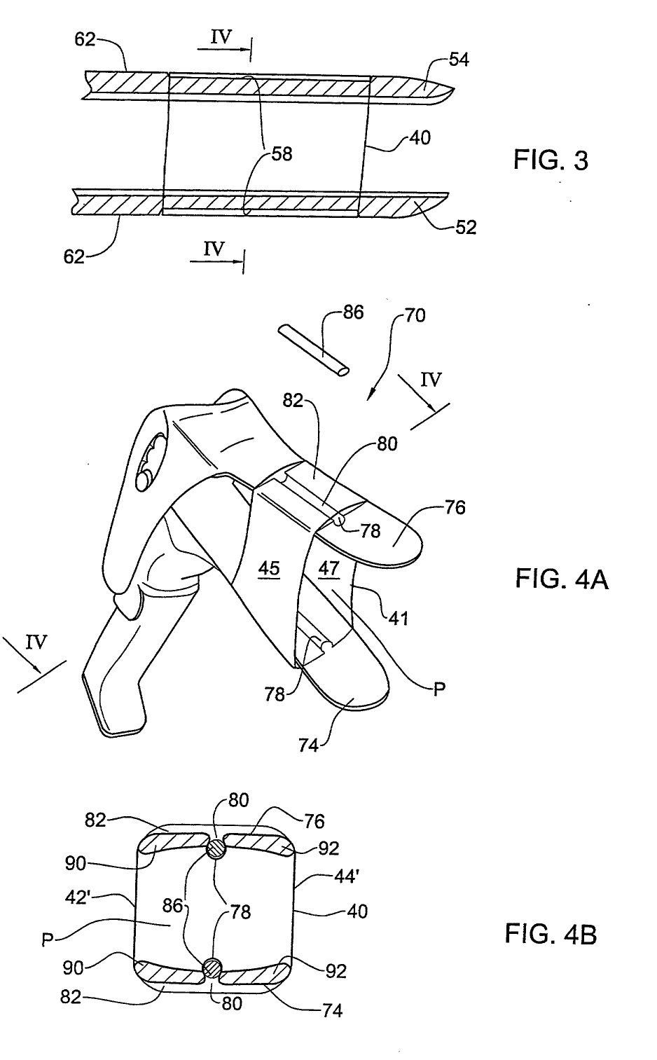

Further attention is now directed to FIG. 2 of the drawings illustrating a speculum 50 having the same general construction as speculum 10 in FIG. 1, the difference residing in the particular design of the lower blade 52 and the upper blade 54, each formed with a depressed central portion 58 (seen also in FIG. 3) whereby the transparent sleeve 41 (similar with sleeve 40 illustrated in FIG. 1) is received within the depressed portion 58, whereby it envelopes the outer surfaces of the blades 52 and 54 being flush with the outermost surfaces 62 and 64, respectively of the blades in order not to interfere with the introducing process of inserting the speculum into the vagina and so as not to cause irritation of the gentle tissue.

In the embodiment of FIGS. 2 and 3, the sleeve 41 is fixedly attached to the blades 52 and 54 by an adhesive member applied at the distal and proximal regions of the sleeve 41 so as to ensure that the sleeve does not disengage or dislocate from the blades during insertion and removal of the device from the vagina.

In the embodiment of FIGS. 4A and 4B the speculum generally designated 70 is principally similar to the speculum illustrated in connection with the embodiment of FIGS. 1 and 2 the difference residing in the arrangement by which the sleeve 43 is attached to the blades 74 and 76. Each of the blades is formed with a groove 78 having a circular cross-section and with a longitudinal aperture 80 extending the length of the indented portion 82 (corresponding with indented portion 58 in FIG. 3).

A rod 86 (seen separately over the upper blade 76) is snapingly inserted into recesses 78 arresting sleeve 43 within the recesses, thus it within the indented portion 82 and preventing it from detaching or dispositioning with respect to the blades 74 and 76.

It is appreciated tat the sleeve 40, may be a prior fitted on the speculum 70 or may be attached thereto upon demand. It is Her noted that the sleeve 40 gives rise to formation of side walls 45 and 47 only when the speculum 70 is in the open position, i.e. tensioned over corresponding side edges 90 and 92 of the lower blade 74 and upper blade 76, respectively (FIG. 4B), whereby a tubular path P is formed.

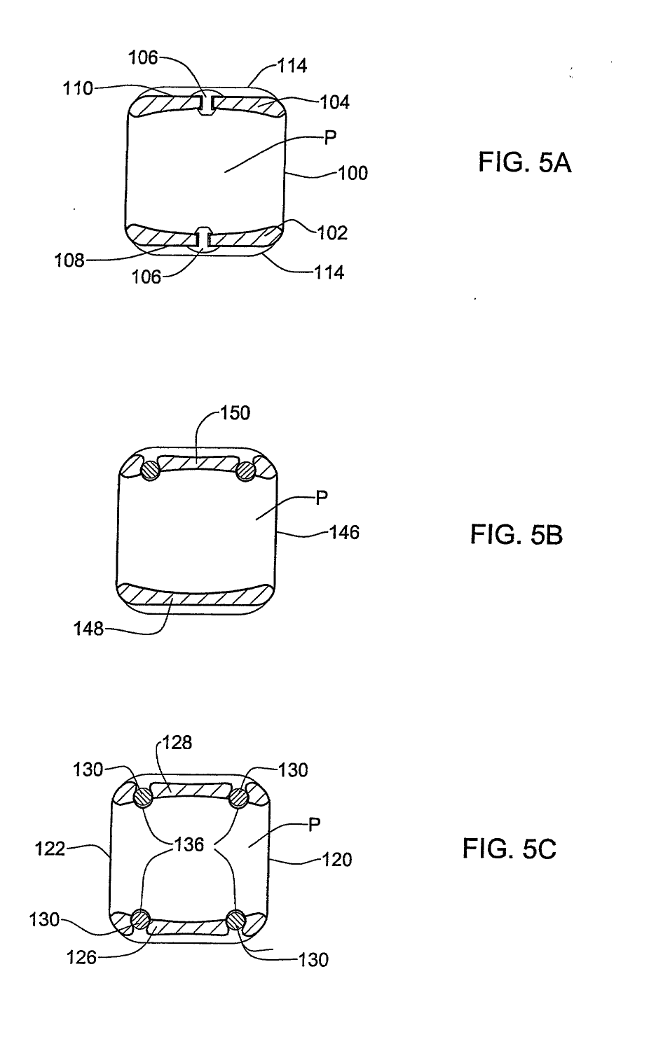

The embodiments of FIGS. 5A to 5C illustrate views corresponding with of FIG. 4B exemplifying further embodiments of securing the film of material to the blades and it is to be appreciated that these are mere examples and other possibilities are applicable as well.

In FIG. 5A, the film is a closed sleeve 100, similar to the previous embodiments, wherein the sleeve is secured to the lower blade 102 and upper blade 104 by a wedge-type fastener 106 snap fitted into the blades through corresponding portions 108 and 110 of the film 100, at those depressed portions of the blades, whereby the wedges do not project beyond the outermost surface 114 of blades 102 and 104.

In the embodiment of FIG. 5B, two sheets of material 120 and 122 are provided, extending between a lower blade 126 and an upper blade 128, said films being secured to the blades 126 and 128 in a similar manner as illustrated in connection with FIGS. 4A and 4B by means of a snap-type rod 130 clampingly securing edges of the films 120 and 122 within corresponding recesses 136 formed in the blades.

In the embodiment of FIG. 5C, a unitary film 146 is provided, enveloping a lower blade 148 and attached to an upper blade 150 in a similar manner as illustrated in connection with FIG. 5B. Optionally, a respective portion of the film 146 enveloping the lower blade 148 may be secured by an adhesive layer.

It is apparent from the figures that deploying the device into it open position gives rise to constitution of side walls which will bear sufficient load and prevent tissue from collapsing into the path formed between the blades and the side walls. This path is designated in FIG. 4B and in FIGS. 5A-5C as P.

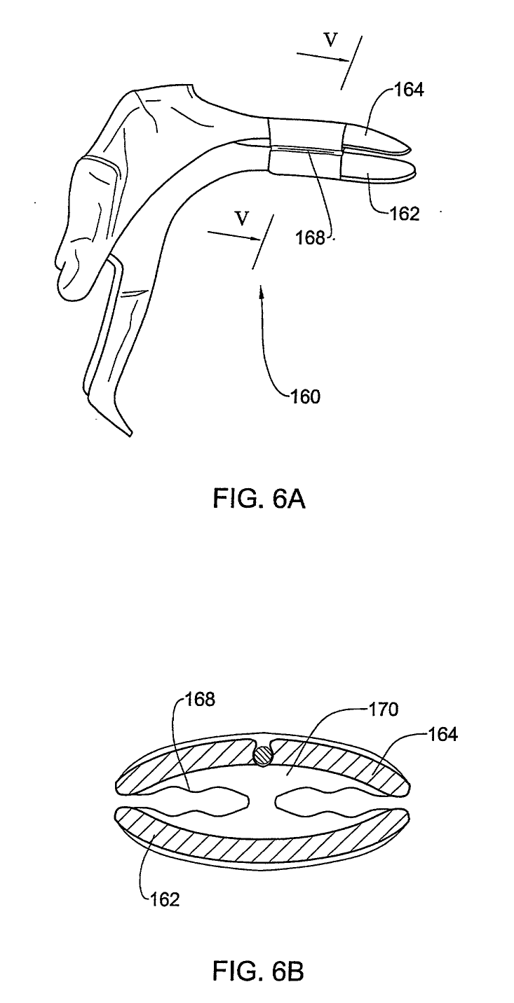

Turning now to FIGS. 6A and 6B, there is illustrated a speculum in accordance with the present invention generally designed 160, the device illustrated in is closed position, i.e. wherein the lower blade 162 and the upper blade 164 are adjacent one another. This is the packaging position of the device as well as the introducing position for inserting it into the vagina. In this position, the film of material 168 is collapsed and received within the confined space 170 extending between the blades.

Whilst the present invention has been described and illustrated with respect to a particular type of vaginal speculum, it is to be appreciated that the invention is not restricted to a vaginal speculum nor is it restricted to the particular detected device. Rather, it is an intention of the invention to cover all modifications and arrangements falling within the spirit and the scope of the invention, mutatis mutandis.

1. A surgical instrument for carrying out diagnostic and therapeutic procedures on interior organs of a body, the instrument comprising a rigid lower blade and a rigid upper blade, coupled so as to be spatially adjustable between a closed position and at least one open position, and a film of flexible material extending from corresponding side edges between the lower blade and the upper blade, which at an open position said film is tensioned thereby constituting two side walls of the instrument.

2. A surgical instrument according to claim 1, wherein the film is in the form of a sleeve enveloping an outer surfaces of both the lower blade and the upper blade.

3. A surgical instrument according to claim 1, wherein the film is a unitary film enveloping an outer surface of one of the lower blade and the upper blade and engaged to the other of the one of lower blade and the upper blade.

4. A surgical instrument according to claim 1, wherein two films are provided, each secured to and extending from corresponding side edges of the lower blade and the upper blade.

5. A surgical instrument according to claim 1, being a disposable vaginal speculum.

6. A surgical instrument according to claim 1, where at least one of the lower blade and the upper blade is formed with an indeed outer surface accommodating the film.

7. A surgical instrument according to claim 1, wherein the film is secured to at least one of the lower blade and the upper blade adjacent a front end and a rear end of the film.

8. A surgical instrument according to claim 1, wherein the film of material is between about 3 to 10 cm wide.

9. A surgical instrument according to claim 1, wherein the film is made of transparent material.

10. A surgical instrument according to claim 1, wherein the film is made of a non-stretchable though pliable material.

11. A surgical instrument according to claim 1, wherein at the closed position the film is collapsed and received within a space between the lower blade and the upper blade.

12. A surgical instrument according to claim 1, wherein at the film is secured to one or both of the lower blade and the upper blade by one or more snap-type members securing the film to the respective blade.

13. A surgical instrument according to claim 1, wherein at the film is secured to one or both of the lower blade and the upper blade by a clamping arrangement wherein a respective blade portion is formed with a locking-receptacle adapted for lockingly arresting a lock-member clamping there between a portion of the film.

14. A film of material mountable on a surgical device used for carrying out diagnostic and therapeutic procedures on interior organs of a body, said device comprising a rigid lower blade and a rigid upper blade coupled so as to be spatially adjustable between a closed position and at least one open position; said film being made of a flexible and non-stretchable material, for constituting two side walls of the device.

15. A surgical instrument for carrying out diagnostic and therapeutic procedures on interior organs of a body, the instrument comprising a rigid lower blade and a rigid upper blade each having longitudinal side edges, a handle associated with one of the blades, the other blade being spatially adjustable; side walls made of a non-stretchable though pliable material, extending between corresponding side edges of the blades and sized such that at an open position said side walls are tensioned thus, constituting together with the blades a tubular portion through which diagnostic and therapeutic procedures may be carried out.

16. A surgical instrument according to claim 15, wherein the film is in the form of a sleeve enveloping an outer surfaces of both the lower blade and the upper blade.

17. A surgical instrument according to claim 15, wherein at least one of the lower blade and the upper blade is formed with an indented outer surface accommodating the film.

18. A surgical instrument according to claim 15, wherein the film is secured to at least one of the lower blade and the upper blade adjacent a front end and a rear end of the film.

19. A surgical instrument according to claim 15, wherein the film is made of transparent material.

20. A surgical instrument according to claim 15, wherein at the closed position the film is collapsed and received within a space between the lower blade and the upper blade.

21. A surgical instrument according to claim 15, wherein the film is secured to one or both of the lower blade and the upper blade by one or more snap-type members securing the film to the respective blade.

22. A surgical instrument according to claim 15, wherein the film is secured to one or both of the lower blade and the upper blade by a clamping arrangement wherein a respective blade portion is formed with a locking-receptacle adapted for lockingly arresting a lock-member clamping there between a portion of the film.

23. A vaginal speculum comprising a rigid lower blade and a rigid upper blade, each comprising two side edges and being articulated in a manner so as to be spatially adjustable between a closed position and at least one open position, and two side walls made of a flexible material extending between corresponding side edges of the blades and sized such that at the open position said side walls are tensioned thus forming together with the blades a tubular portion, through which diagnostic and therapeutic procedures may be carried outs and where at a closed position said walls are collapsed and received within a space between the lower blade and the upper blade.

24. A vaginal speculum according to claim 23, wherein the film is in the form of a sleeve enveloping an outer surfaces of both the lower blade and the upper blade.

25. A vaginal speculum according to claim 23, wherein the film is a unitary film enveloping an outer surface of one of the lower blade and the upper blade and engaged to the other of the one of lower blade and the upper blade.

26. A Dual speculum according to claim 23, wherein at least one of the lower blade and the upper blade is formed with an indented outer surface accommodating the film.

27. A vaginal speculum according to claim 23, wherein the film is secured to at least one of the lower blade and the upper blade adjacent a front end and a rear end of the film.

28. A vaginal speculum according to claim 23 wherein the film of material is between about 3 to 10 cm wide.

29. A vaginal speculum according to claim 23, wherein the film is made of transparent material.

30. A vaginal speculum according to claim 23, who at the closed position the film is collapsed and received within a space between the lower blade and the upper blade.

31. A vaginal speculum according to claim 23, wherein the film is secured to one or both of the lower blade and the upper blade by one or more snap-type members securing the film to the respective blade.

32. A vaginal speculum according to claim 23, wherein the film is secured to one or both of the lower blade and the upper blade by a clamping arrangement wherein a respective blade portion is formed with a locking-receptacle adapted for lockingly arresting a lock-member clamping there between a portion of the film.

33. A vaginal speculum according to claim 23, wherein the film is made of a non-stretchable though pliable material at the open state of the device the side walls prevent the vagina's walls from entering into the space between the blades.

34. A film of material for use with a device according to any one of claims 1, 15 and 23.
